(19) United States
(12) Patent Application Publication
(10) Pub. No.: US 2005/0182437 A1
(43) Pub. Date: Aug. 18, 2005

(54) Guidewire assembly including a repeatably inflatable occlusive balloon on a guidewire ensheathed with a spiral coil

(76) Inventors: Michael J. Bonnette, Minneapolis, MN (US); Eric J. Thor, Arden Hills, MN (US); Richard R. Prather, St. Michael, MN (US)

Correspondence Address: HUGH D JAEGER, 1000 SUPERIOR BLVD, SUITE 302, WAYZATA, MN, 553911873, US

(21) Appl. No.: 10/838,468

(22) Filed: May 4, 2004

Related U.S. Application Data
(63) Continuation-in-part of application No. 10/012,903, filed on Nov. 6, 2001.
(63) Continuation-in-part of application No. 10/012,891, filed on Nov. 6, 2001.
(63) Continuation-in-part of application No. 10/007,788, filed on Nov. 6, 2001.

(51) Int. Cl. A61M 29/00; A61M 25/00

(52) U.S. Cl. 606/194

(57) ABSTRACT

A guidewire assembly including a repeatably inflatable occlusive balloon on a guidewire ensheathed in a close wound spiral coil. The guidewire assembly is used as part of a guidewire occlusion system.

### CROSS REFERENCES TO RELATED APPLICATIONS

This application is a continuation-in-part of U.S. patent application Ser. No. 10/012,903, filed Nov. 6, 2001, entitled “Guidewire Occlusion System Utilizing Repeatably Inflatable Gas-Filled Occlusive Device,” and U.S. patent application Ser. No. 10/012,891, filed Nov. 6, 2001, entitled “Guidewire Assembly Having Occlusive Device and Repeatably Crimpable Proximal End,” and U.S. patent application Ser. No. 10/007,788, filed Nov. 6, 2001, entitled “Gas Inflation/Evacuation System and Sealing System for Guidewire Assembly Having Occlusive Device,” all of which are hereby incorporated herein by reference.

Also related to the instant application is commonly owned patent application Ser. No. ______, entitled “Gas Inflation/Evacuation System and Sealing System Incorporating A Compression Sealing Mechanism for Guidewire Assembly Having Occlusive Device”, filed on even date herewith. This application too is hereby incorporated herein by reference.

### BACKGROUND OF THE INVENTION

1. Field of the Invention

The present invention relates generally to the field of vascular medical devices. More specifically, the present invention relates to a guidewire assembly including a repeatably inflatable occlusive balloon on a guidewire ensheathed with a spiral coil for use as part of a guidewire occlusion system in vascular procedures.

2. Description of the Prior Art

Arterial disease involves damage that happens to the arteries in the body. Diseased arteries can become plugged with thrombus, plaque, or grumous material that may ultimately lead to a condition known as ischemia. Ischemia refers to a substantial reduction or loss of blood flow to the heart muscle or any other tissue that is being supplied by the artery and can lead to permanent damage of the affected region. While arterial disease is most commonly associated with the formation of hard plaque and coronary artery disease in the heart, similar damage can happen to many other vessels in the body, such as the peripheral vessels, cerebral vessels, due to the buildup of hard plaque or softer thrombus or grumous material within the lumen of an artery or vein.

A variety of vascular medical devices and procedures have been developed to treat diseased vessels. The current standard procedures include bypass surgery (where a new blood vessel is grafted around a narrowed or blocked artery) and several different types of non-surgical interventional vascular medical procedures, including angioplasty (where a balloon on a catheter is inflated inside a narrowed or blocked portion of an artery in an attempt to push back plaque or thrombotic material), stenting (where a metal mesh tube is expanded against a narrowed or blocked portion of an artery to hold back plaque or thrombotic material), and debulking techniques in the form of atherectomy (where some type of high speed or high power mechanism is used to dislodge hardened plaque) or thrombectomy (where some type of mechanism or infused fluid is used to dislodge grumous or thrombotic material). In each of these interventional vascular medical procedures, a very flexible guidewire is routed through the patient's vascular system to a desired treatment location and then a catheter that includes a device on the distal end appropriate for the given procedure is tracked along the guidewire to the treatment location.

The prior art guidewires suffer from several drawbacks. One drawback is that the size is often too small to give enough support to larger peripheral devices that need to be passed thereover. For example, the majority of guidewires used in the aforementioned interventional procedures have a maximum diametric dimension of 0.014 inch, and such is not sufficient to lend proper support to certain devices that need to be passed thereover. Larger guidewires are known, e.g., guidewires having a diametric dimension as large as 0.035 inch, but those guidewires have poor steerability. Also, prior art guidewires of this size have not been known to have an occlusive device, in particular an occlusive balloon, thereon.

The use of an occlusive device as part of a vascular procedure is becoming more common in debulking procedures performed on heart bypass vessels. Most heart bypass vessels are harvested and transplanted from the saphenous vein located along the inside of the patient's leg. The saphenous vein is a long, straight vein that has a capacity more than adequate to support the blood flow needs of the heart. Once transplanted, the saphenous vein is subject to a buildup of plaque or thrombotic materials in the grafted arterial lumen. Unfortunately, the standard interventional vascular treatments for debulking are only moderately successful when employed to treat saphenous vein coronary bypass grafts. The complication rate for a standard balloon angioplasty procedure in a saphenous vein coronary bypass graft is higher than in a native vessel with the complications including embolization, “no-reflow” phenomena, and procedural related myocardial infarction. Atherectomy methods including directional, rotational, and laser devices are also associated with a high degree of embolization resulting in a greater likelihood of infarction. The use of stents for saphenous vein coronary bypass grafts has produced mixed results. Stents provide for less restenosis, but they do not eliminate the risk of embolization and infarction incurred by standard balloon angioplasty.

In order to overcome the shortcomings of these standard non-surgical interventional treatments in treating saphenous vein coronary bypass graft occlusion, embolic protection methods utilizing a protective device distal to the lesion have been developed. The protective device is typically a filter or a balloon. Use of a protective device in conjunction with an atherectomy or thrombectomy device is intended to prevent emboli from migrating beyond the protective device and to allow the embolic particles to be removed, thereby subsequently reducing the risk of myocardial infarction. When the occlusive device is a balloon, the balloon is inserted and inflated at a point distal to the treatment site or lesion site. Therapy is then performed at the treatment site and the balloon acts to block all blood flow which prevents emboli from traveling beyond the balloon. Following treatment, some form of particle removal device must be used to remove the dislodged emboli prior to balloon deflation. U.S. Pat. No. 5,843,022 uses a balloon to occlude the vessel distal to a lesion or blockage site. The occlusion is treated with a high pressure water jet, and the fluid and entrained emboli are subsequently removed via an extraction tube. U.S. Pat. No. 6,135,991 describes the use of a balloon to occlude the vessel allowing blood flow and pressure to prevent the migration of emboli proximally from the treatment device.

There are various designs that have included an occlusive balloon on the end of a guidewire. U.S. Pat. Nos. 5,520,645, 5,779,688 and 5,908,405 describe guidewires having removable occlusive balloons on a distal end. U.S. Pat. No. 4,573,470 describes a guidewire having an occlusive balloon where the guidewire is bonded inside the catheter as an integral unit. U.S. Pat. Nos. 5,059,176, 5,167,239, 5,520,645, 5,779,688 and 6,050,972 describe various guidewires with balloons at the distal end in which a valve arrangement is used to inflate and/or deflate the balloon. U.S. Pat. No. 5,908,405 describes an arrangement with a removable balloon member that can be repeatedly inserted into and withdrawn from a guidewire. U.S. Pat. No. 5,776,100 describes a guidewire with an occlusive balloon adhesively bonded to the distal end with an adapter on the proximal end to provide inflation fluid for the occlusive balloon.

Except in the case of the normal cerebral anatomy where there are redundant arteries supplying blood to the same tissue, one of the problems with using an occlusive device in the arteries is that tissue downstream of the occlusive device can be damaged due to the lack of blood flow. Consequently, an occlusive device that completely blocks the artery can only be deployed for a relatively short period of time. To overcome this disadvantage, most of the recent development in relation to occlusive devices has focused on devices that screen the blood through a filter arrangement. U.S. Pat. Nos. 5,827,324, 5,938,672, 5,997,558, 6,080,170, 6,171,328, 6,203,561 and 6,245,089 describe various examples of filter arrangements that are to be deployed on the distal end of a catheter system. While a filter arrangement is theoretically a better solution than an occlusive device, in practice such filter arrangements often become plugged, effectively turning the filter into an occlusive device. The filter arrangements also are mechanically and operationally more complicated than an occlusive balloon device in terms of deployment and extraction.

As is the case in almost all angioplasty devices or stenting catheter devices where a balloon is used to expand the blood vessel or stent, most catheter occlusive balloons as well as most guidewire occlusive balloons utilize a liquid fluid such as saline or saline mixed with a radiopaque marker for fluoroscopic visualization (i.e., contrast) as the inflation medium. Generally, a liquid fluid medium for expanding vascular balloons has been preferred because the expansion characteristics of a liquid are more uniform and predictable, and because a liquid medium is easier to work with and more familiar to the doctors. In the case of angioplasty balloons, for example, high-pressure requirements (up to 20 atmospheres) necessitate that the inflation fluid be an incompressible fluid for safety reasons. While having numerous advantages, liquid fluids do not lend themselves to rapid deflation of an occlusive balloon because of the high resistance to movement of the liquid in a long small diameter tube. In the context of angioplasty procedures, the balloon catheter has a much larger lumen than a guidewire. Consequently, rapid deflation is possible. In the context of a guidewire, however, liquid filled occlusive balloons typically cannot be deflated in less than a minute and, depending upon the length of the guidewire, can take up to several minutes to deflate. Consequently, it is not practical to shorten the period of total blockage of a vessel by repeatedly deflating and then re-inflating a liquid filled occlusive balloon at the end of a guidewire.

Gas-filled balloons have been used for intra-aortic occlusive devices where rapid inflation and deflation of the occlusive device is required. Examples of such intra-aortic occlusive devices are shown in U.S. Pat. Nos. 4,646,719, 4,733,652, 5,865,721, 6,146,372, 6,245,008 and 6,241,706. While effective for use as an intra-aortic occlusive device, these occlusive devices are not designed for use as a guidewire as there is no ability to track a catheter over the intra-aortic occlusive device.

An early catheter balloon device that utilized a gas as an inflation medium and provided a volume limited syringe injection system is described in U.S. Pat. No. 4,865,587. More recently, a gas-filled occlusive balloon on a guidewire is described as one of the alternate embodiments in U.S. Pat. No. 6,217,567. The only suggestion for how the guidewire of the alternate embodiment is sealed is a valve type arrangement similar to the valve arrangement used in a liquid fluid embodiment. A similar gas-filled occlusive balloon has been described with respect to the Aegis Vortex™ system developed by Kensey Nash Corporation. In both U.S. Pat. No. 6,217,567 and the Aegis Vortex™ system, the gas-filled occlusive balloon is used for distal protection to minimize the risk of embolization while treating a blocked saphenous vein coronary bypass graft. Once deployed, the occlusive balloon retains emboli dislodged by the atherectomy treatment process until such time as the emboli can be aspirated from the vessel. No specific apparatus are shown or described for how the gas is to be introduced into the device or how the occlusive balloon is deflated.

Although the use of occlusive devices has become more common for distal embolization protection in vascular procedures, particularly for treating a blocked saphenous vein coronary bypass graft, all of the existing approaches have significant drawbacks that can limit their effectiveness. Liquid filled occlusive balloons can remain in place too long and take too long to deflate, increasing the risk of damages downstream of the occlusion. Occlusive filters are designed to address this problem, but suffer from blockage problems and can be complicated to deploy and retrieve and may allow small embolic particles to migrate downstream. Existing gas-filled occlusive balloons solve some of the problems of liquid filled occlusive balloons, but typically have utilized complicated valve and connection arrangements. It would be desirable to provide for an occlusive device that was effective, simple, quick to deploy and deflate, and that could overcome the limitations of the existing approaches.

Some of these problems have been previously addressed in three commonly owned and assigned co-pending applications, which are hereby incorporated by reference herein: “Guidewire Occlusion System Utilizing Repeatably Inflatable Gas-Filled Occlusive Device,” application Ser. No. 10/012,903, filed Nov. 6, 2001; “Guidewire Assembly Having Occlusive Device and Repeatably Crimpable Proximal End,” application Ser. No. 10/012,891, filed Nov. 6, 2001; and “Gas Inflation/Evacuation System and Sealing System for Guidewire Assembly Having Occlusive Device,” application Ser. No. 10/007,788, filed Nov. 6, 2001.

### SUMMARY OF THE INVENTION

This invention is directed to a guidewire assembly which can be utilized as an alternative to those guidewire assemblies disclosed in the three co-pending applications mentioned in the immediately preceding paragraph as well as that guidewire assembly disclosed in the aforementioned application filed on even date herewith. The guidewire assembly disclosed herein involves a repeatably inflatable occlusive device in the form of an occlusive balloon mounted on a guidewire ensheathed with a spiral coil. The ensheathed guidewire has an outer diameter ranging from 0.030 inch to 0.040 inch, preferably 0.035 inch, thus making it more robust than the guidewires of the above-mentioned applications such that it is strong enough to support the various surgical procedure devices that it is desired to employ with it. The spiral coil provides the guidewire with good steerability.

One significant aspect and feature of the present invention is a guidewire assembly which is able to adequately support surgical devices to be passed thereover and yet still be easily maneuverable.

Another significant aspect and feature of the present invention is a guidewire that is surrounded by a spiral coil having close wound, abutting turns which imparts good steerability to the guidewire.

Still another significant aspect and feature of the present invention is a guidewire that is surrounded by a spiral coil which is secured to the guidewire by solder at spaced intervals along the length of the guidewire.

Yet another significant aspect and feature of the present invention is a guidewire having a spiral coil therearound along its entire length except for a balloon mounting portion and an extended sealable section.

Having thus briefly described the present invention and mentioned some of the significant aspects and features thereof, it is the principal object of the present invention to provide a guidewire assembly including a repeatably inflatable occlusive balloon on a guidewire ensheathed with a spiral coil.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1 is a schematic diagram of a guidewire occlusion system operating in an evacuation mode;

FIG. 2 is a schematic diagram of the guidewire occlusion system shown in FIG. 1 operating in an inflation mode;

FIG. 3 is a side view of the guidewire assembly in accordance with the present invention, the spiral coil being drawn in open pitch for visualization of underlying structure;

FIG. 4 is a longitudinal cross-sectional view of the guidewire assembly shown in FIG. 3;

FIG. 4a is an enlarged view of the right-hand portion of FIG. 4; and,

FIG. 5 is an enlarged view of the distal end portion of the guidewire assembly shown in FIG. 3.

### DETAILED DESCRIPTION OF THE PREFERRED EMBODIMENTS

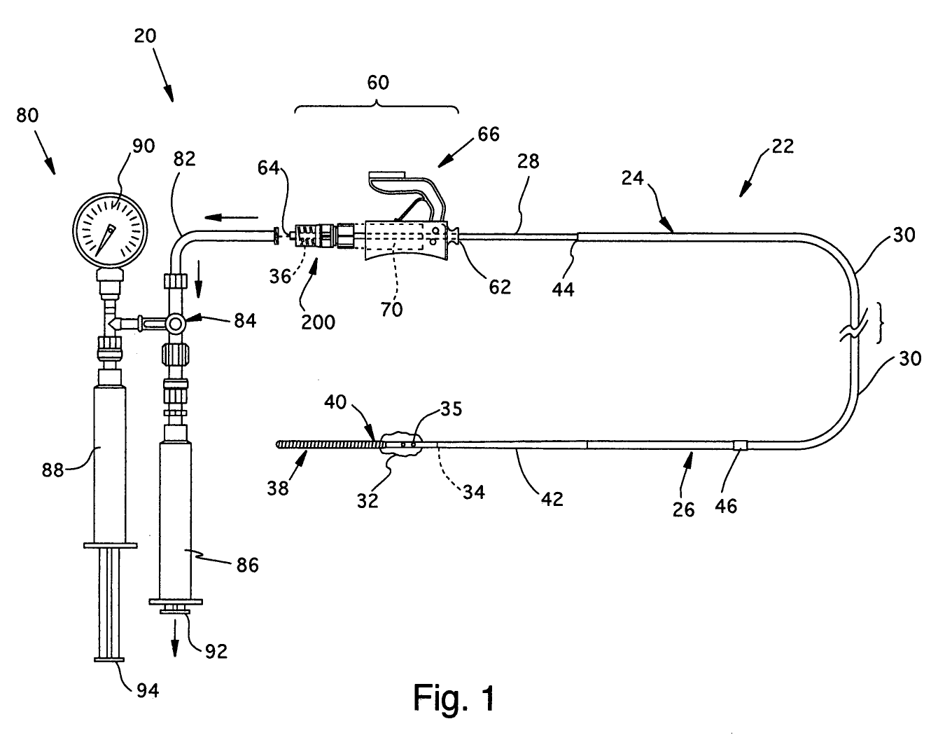

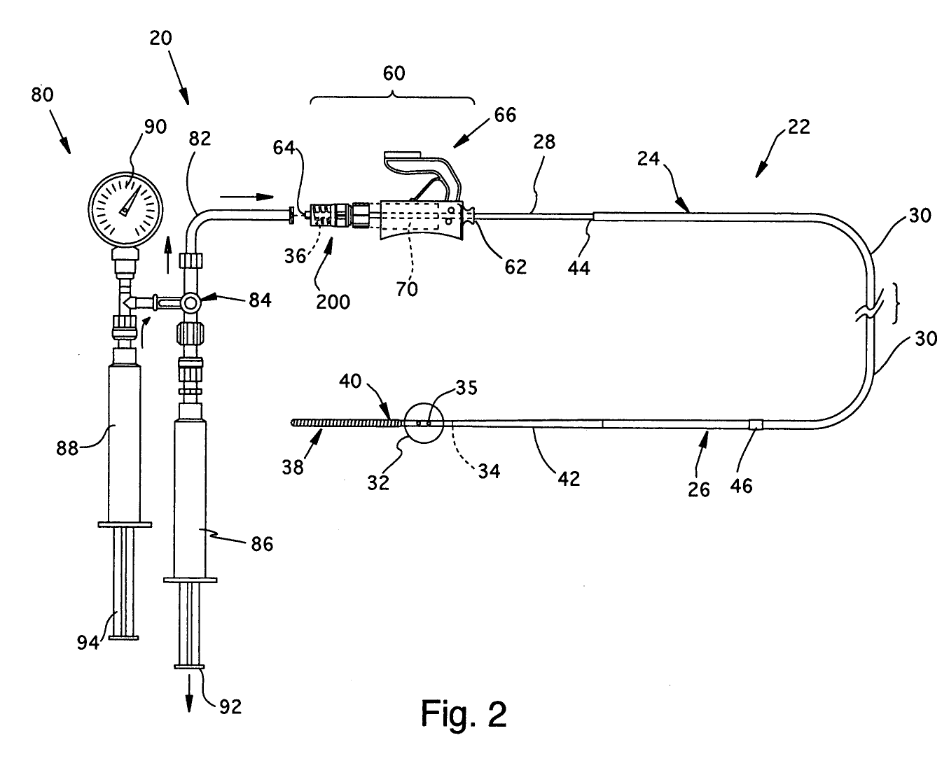

Referring now to FIGS. 1 and 2, the overall structure and operation of a guidewire occlusion system 20 will be described. The guidewire occlusion system 20 includes a guidewire assembly 22, a sealing system 60, and a gas inflation/evacuation system 80. The preferred embodiments of the overall guidewire occlusion system 20 are described in further detail in the previously identified co-pending applications entitled “Guidewire Occlusion System Utilizing Repeatably Inflatable Gas-Filled Occlusive Device”, “Guidewire Assembly Having Occlusive Device and Repeatably Crimpable Proximal End,”, and “Gas Inflation/Evacuation System and Sealing system for Guidewire Assembly Having Occlusive Device”.

Guidewire assembly 22 includes a guidewire 24, an occlusive device such as an occlusive balloon 32, and, optionally, a flexible tip 38. The guidewire 24 is tubular and comprises an extended sealable section 28, a main body portion 30, and a distal portion 26. Extended sealable section 28 is generally a separate piece which extends from the proximal end 36 of the guidewire 24 to the main body portion 30 to which it is joined, preferably by a laser weld 44. The distal portion 26 also is generally a separate piece which is joined to the main body portion 30, preferably by an Ni—Ti or stainless steel sleeve 46, and extends distally from the main body portion 30 to the distal end 40 of the guidewire 24. As used herein, the terms proximal and distal will be used with reference to an operator, such that a distal portion of the guidewire 24, for example, is the portion first inserted into a blood vessel, and the proximal portion remains exterior to the patient and is therefore closer to the operator. Preferably, the extended sealable section 28 is an extended crimpable section comprised of a tubular segment having an outer diameter smaller than an outer diameter of the main body portion 30 of guidewire 24. Although the diameter of the extended crimpable section could be any size consistent with effective use as a guidewire, it will be understood that the smaller diameter allows for less force to be used in sealing the extended crimpable section and provides a crimped seal that is not too large when crimped. The occlusive balloon 32 is located along the distal portion 26 of guidewire 24. The occlusive balloon 32 is fluidly connected via a lumen 34 to the proximal end 36 of guidewire 24, with channels or holes 35 allowing for fluid communication between lumen 34 and occlusive balloon 32. In a preferred embodiment, the flexible tip 38 is used and it is positioned at the distal end of the guidewire assembly 22. Preferably, distal portion 26 of guidewire 24 includes a tapered portion 42 to increase the flexibility and transition properties of the distal portion 26.

Preferably, sealing system 60 is implemented as part of a handheld apparatus that also includes gas inflation/evacuation system 80. Alternatively, sealing system 60 may be a handheld unit completely separate from the gas inflation/evacuation system 80. Sealing system 60 includes a first aperture 62 into which the proximal end 36 of the guidewire 24 is insertable so as to operably position at least a portion of the extended sealable section 28 in relation to sealing system 60. Sealing system 60 further includes a second aperture 64 that is fluidly connectable to gas inflation/evacuation system 80 by a conduit 82. The sealing system 60 includes means for selectively sealing the extended sealable section 28 which in the preferred embodiment comprises a crimping mechanism 66 and a compression sealing mechanism 200. A passageway 70 is defined from first aperture 62 to second aperture 64 and extends through both crimping mechanism 66 and compression sealing mechanism 200. A portion of the extended sealable section 28 is inserted into first aperture 62 a sufficient distance to engage crimping mechanism 66 and compression sealing mechanism 200. The crimping mechanism 66 and the compression sealing mechanism 200 are described in detail in the previously mentioned patent application Ser. No. ______ filed concurrently herewith.

The gas inflation/evacuation system 80 is connected via conduit 82 to the second aperture 64 of the sealing system 60. The gas inflation/evacuation system 80 preferably includes a valve arrangement 84 that selectively couples one of an evacuation system which includes means for evacuating the guidewire assembly 22 and an inflation system which includes means for introducing a gas into the guidewire assembly 22 to the conduit 82. The evacuation system includes an evacuation syringe 86 having a plunger 92 which is used to evacuate the guidewire assembly 22, passageway 70, and conduit 82. The inflation system includes an inflation syringe 88 having a plunger 94 which contains a volume of a biocompatible gas sufficient to inflate the occlusive balloon 32 a plurality of times. Preferably, the biocompatible gas is carbon dioxide. Other biocompatible gasses that may be utilized with the present invention include oxygen, nitrogen, and nitrous oxide. Optionally, a pressure gauge 90 can be associated with the inflation syringe 88.

In a preferred embodiment, the main body portion 30 is formed of a stainless steel hypotube having an outer diameter of 0.013 inch and an inner diameter of 0.007 inch. To accomplish passive deflation in the desired time of less than one minute when the extended sealable section 28 is cut, it is preferable that the main body portion 30 have an inner diameter of at least 0.002 inch. The extended sealable section 28 of guidewire 24 is comprised of a crimp tube also formed of stainless steel and having an outer diameter of 0.009 inch to 0.015 inch and an inner diameter of at least 0.002 inch and preferably about 0.005 inch. As mentioned before, the extended sealable section 28 is generally a separate piece secured to the main body portion 30 by a laser weld 44. Alternatively, the extended sealable section 28 may be formed by centerless grinding or reducing the outer diameter of a portion of the proximal portion of the main body portion 30 of guidewire 24. Still other embodiments may enable the extended sealable section to be a modified, treated or otherwise fabricated portion of the proximal portion of the main body portion 30 that is suitable for the particular sealing technique to be used.

The extended sealable section 28 can be made of any material that when deformed and severed retains that deformation so as to form an airtight seal. When crimped and severed, it is preferable that the extended sealable section 28 not present a sharp, rigid point that is capable of piercing a gloved hand. It has been found that so long as the preferred embodiment is not gripped within less than one inch of the proximal end of the extended sealable section 28, the severed proximal end of the extended sealable section 28 does not penetrate a standard surgical glove. In addition, the extended sealable section 28 must have sufficient strength in terms of high tensile and kink resistance to permit catheter devices to repeatedly pass over the extended sealable section 28.

The main body portion 30 is preferably secured to the distal portion 26 using a Ni—Ti or stainless steel sleeve 46 laser welded to the main body portion 30 and crimped to the distal portion 26. The distal portion 26 is preferably formed of a Ni—Ti alloy having an inner diameter of 0.0045 inch and an outer diameter that ranges from 0.014 inch to 0.0075 inch to form tapered portion 42, preferably formed by a centerless grinding process. The flexible tip 38 is a coiled tip attached to distal portion 26 distal to occlusive balloon 32, preferably by crimping. Alternatively, a sleeve could be welded to the flexible tip 38, and the tapered portion 42 could then be inserted into this sleeve and crimped.

Alternatively, any number of other alloys or polymer materials and attachment techniques could be used in the construction of the guidewire 24, provided the materials offer the flexibility and torque characteristics required for a guidewire and the attachment techniques are sufficiently strong enough and capable of making an airtight seal. These materials include, but are not limited to, Ni—Ti, 17-7 stainless steel, 304 stainless steel, cobalt superalloys, or other polymer, braided or alloy materials. The attachment techniques for constructing guidewire 24 include, but are not limited to, welding, mechanical fits, adhesives, sleeve arrangements, or any combination thereof.

The occlusive balloon 32 may be made of any number of polymer or rubber materials. Preferably, the occlusive balloon is preinflated to prestretch it so that expansion is more linear with pressure. Preferably, the pressure supplied by gas inflation/evacuation system 80 is designed to stay well within the elastic limit of the occlusive balloon 32. A two-layer occlusive balloon arrangement, adding gas and/or liquid between balloon layers, may be used in an alternate embodiment to increase visibility of the distal end 40 of the guidewire 24 under fluoroscopy.

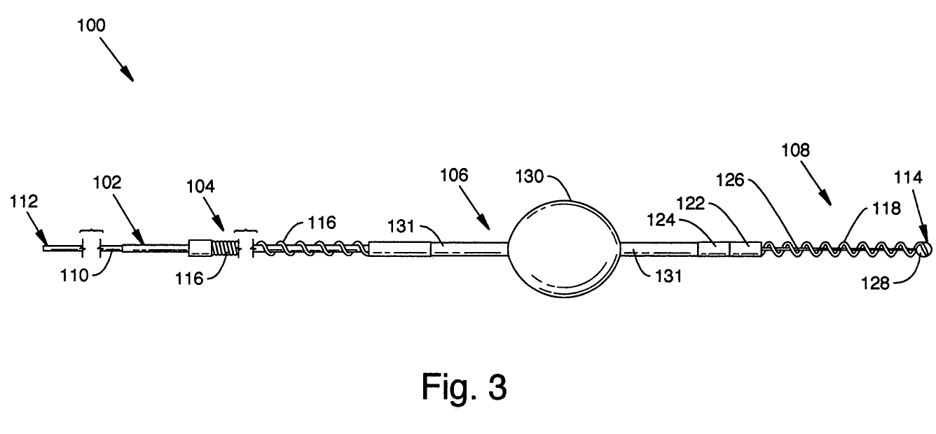

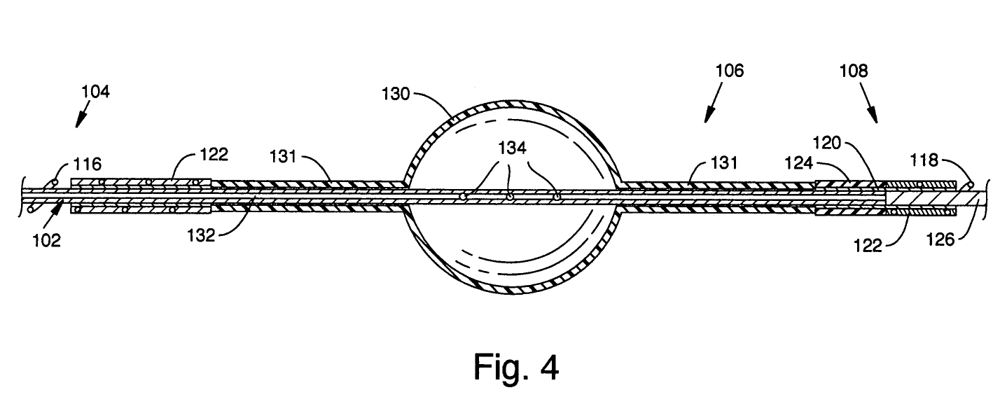

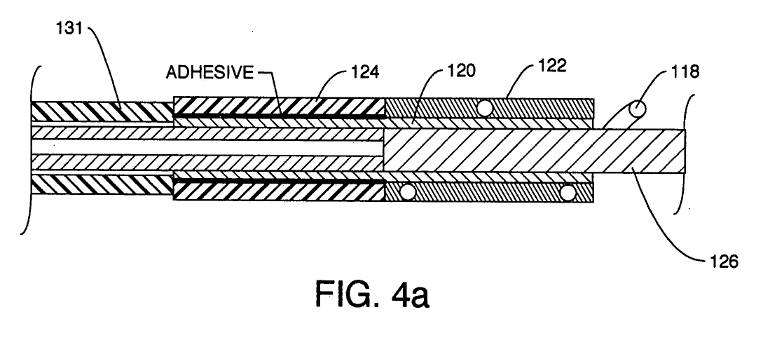

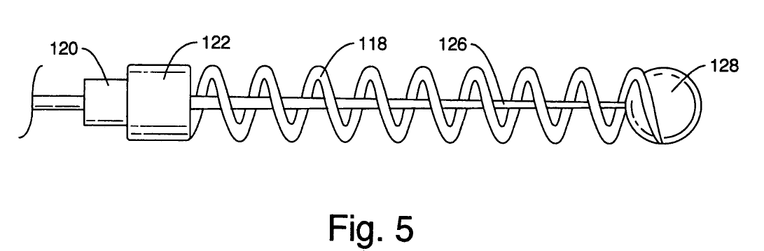

The present invention is now described with reference to FIGS. 3-5. Therein is shown a guidewire assembly 100 which is an alternative embodiment to the guidewire assembly 22 depicted in FIGS. 1 and 2 and which is used in lieu of the guidewire assembly 22 when a stronger, more robust guidewire assembly is needed.

The alternative guidewire assembly 100 comprises a guidewire 102 having a proximal portion 104, a balloon mounting portion 106, and a distal portion 108, and an extended sealable section 110 which is crimpable like the extended sealable section 28. Guidewire 102 has a proximal end 112 and a distal end 114. Mounted over the guidewire 102 at the proximal portion 104 is a first close wound spiral coil 116 which extends from the extended sealable section 110 to the balloon mounting portion 106. The first close wound spiral coil 116 is illustrated in part with open pitch to enable visualization of underlying components, but it is to be understood that it is actually wound as shown at the extreme left of the proximal portion 104. Similarly, mounted over the guidewire 102 along the distal portion 106 is a second close wound spiral coil, again shown distended to enable visualization of underlying structure. The first and second close wound spiral coils 116 and 118 are affixed to the guidewire 102 by solder masses 122 spaced at intervals along the guidewire 102. At the distal portion a solder mass 122 is first secured to a stainless steel sleeve 120 which has a polyimide sleeve also secured to it. The polyimide sleeve is optional. Mounted over the balloon mounting portion 106 is an occlusive balloon 130 which has end portions 131 that tightly embrace and seal to the guidewire 102. A lumen 132 extends from the proximal end 112 to the balloon mounting portion 106 and communicated with the interior of the occlusive balloon via at least one opening or hole 134. The first and second close wound spiral coils enhance the steerability of the guidewire assembly 100 and may be coated with polytetrafluoroethylene to aid in their passage through a blood vessel. Also, proximal of the balloon mounting portion is a zone for receiving a torque device for torqueing the guidewire assembly. The distal portion 108 includes a tapered portion 126 having a bulbous member 128 on its end.

The guidewire assembly 100 functions exactly like the guidewire assembly 22 in the guidewire occlusion system 20. Reference may be had to the aforementioned patent application filed concurrently herewith for a detailed explanation of the mode of operation.

Various modifications can be made to the present invention without departing from the apparent scope thereof.

### Parts List

20 guidewire occlusion system
22 guidewire assembly
24 guidewire
26 distal portion
28 extended sealable section
30 main body portion
32 occlusive balloon
34 lumen
35 channel or hole
36 proximal end
38 flexible tip
40 distal end
42 tapered portion
44 laser weld
46 Ni—Ti or stainless steel sleeve
60 sealing system
62 first aperture
64 second aperture
66 crimping mechanism
70 passageway
80 gas inflation/evacuation system
82 conduit
84 valve arrangement
86 evacuation syringe
88 inflation syringe
90 pressure gauge
92 evacuation syringe plunger
94 inflation syringe plunger
100 guidewire assembly
102 guidewire
104 proximal portion
106 balloon mounting portion
108 distal portion
110 extended sealable section
112 proximal end
114 distal end
116 first close wound spiral coil
118 second close wound spiral coil
120 stainless steel sleeve
122 solder mass
124 polyimide sleeve
126 tapered portion
128 bulbous member
130 occlusive balloon
131 end portion
132 lumen
134 hole
200 compression sealing mechanism

1. A guidewire assembly comprising:
a. a guidewire having,
(1) a proximal portion;
(2) a distal portion having a distal end;
(3) a balloon mounting portion between the proximal portion and the distal portion;
(4) an extended sealable section proximate the proximal portion and having a proximal end;
(5) a lumen extending from the proximal end of the extended sealable section to the balloon mounting portion; and,
(6) at least one opening in the balloon mounting portion in communication with the lumen;
b. an occlusive balloon mounted on the balloon mounting portion over the at least one opening;
c. a first close wound spiral coil surrounding the proximal portion; and,
d. a second close wound spiral coil surrounding the distal portion.

2. The guidewire assembly of claim 1, wherein the balloon mounting portion has a circular cross section.

3. The guidewire assembly of claim 1, wherein the first and second close wound coils are coated with polytetrafluoroethylene.

4. The guidewire assembly of claim 1, wherein the first and second close wound coils are concentrically located about and affixed to the guidewire by masses of solder.

5. The guidewire assembly of claim 1, further comprising a zone located proximal to the occlusive balloon for receiving a torque device for torqueing the guidewire assembly.

6. The guidewire assembly of claim 1, wherein at least a portion of the guidewire is made from a stainless steel and cobalt alloy.

7. The guidewire assembly of claim 1, wherein at least a portion of the guidewire assembly comprises a Ni—Ti alloy.

8. The guidewire assembly of claim 1, wherein the diameter of the guidewire including the surrounding close wound first and second spiral coils is in the range of 0.030 inch to 0.040 inch.

9. The guidewire assembly of claim 8, wherein the diameter is 0.035 inch.

10. The guidewire assembly of claim 1, wherein the distal end comprises a bulbous member.

11. The guidewire assembly of claim 1, wherein the distal portion tapers decreasingly from the balloon mounting portion to the distal end.
